(19) United States
(12) Patent Application Publication
(10) Pub. No.: US 2025/0064306 A1
(43) Pub. Date: Feb. 27, 2025

(54) CAPSULE ENDOSCOPE IMAGING DEVICE, METHOD, AND CAPSULE ENDOSCOPE

(71) Applicant: Ankon Medical Technologies(Shanghai) Co., LTD, Shanghai (CN)

(72) Inventors: Xiaodong DUAN, Plano, TX (US); Qinghu YOU, Shanghai (CN)

(73) Assignees: Ankon Medical Technologies(Shanghai) Co., LTD., Shanghai, CN (CN); ANX IP HOLDING PTE. LTD., SG, SG (SG)

(21) Appl. No.: 18/722,552

(22) PCT Filed: Dec. 20, 2022
(86) PCT No.: PCT/CN2022/140450

§ 371 Date: Jun. 20, 2024

(30) Foreign Application Priority Data
Dec. 20, 2021 (CN) 202111565946.9

(51) Int. Cl. A61B 1/04; A61B 1/06

(52) U.S. Cl. CPC A61B 1/041; A61B 1/0684

(57) ABSTRACT

The present invention discloses a capsule endoscope imaging device, an imaging method, and a capsule endoscope. The imaging device includes first lamp bodies, second lamp bodies, third lamp bodies, a first control circuit, a second control circuit, and a third control circuit. The first lamp bodies emit white light, the second lamp bodies emit light with a central wavelength of 510 nm-540 nm, and the third lamp bodies emit light with a central wavelength of 400 nm-420 nm. The first control circuit, second control circuit, and third control circuit are respectively used to adjust exposure time or voltage of the first lamp bodies, the second lamp bodies, and the third lamp bodies.

### CROSS-REFERENCE OF RELATED APPLICATIONS

The application is a National Phase Application of PCT International Application No. PCT/CN2022/140450, International Filing Date Dec. 20, 2022, published Jun. 29, 2023 as International Publication Number WO2023/116725A1, which claims priority from Chinese Patent Application No. 202111565946.9, filed Dec. 20, 2021, all of which are incorporated herein by reference in their entireties.

### FIELD OF INVENTION

The present invention relates to the field of capsule endoscope technology, in particular to a capsule endoscope imaging device, an imaging method and a capsule endoscope.

### BACKGROUND

Currently, the commonly used gastroscopy technology has developed relatively maturely, offering multiple imaging modes, including White Light Imaging (abbreviated as WLI) mode and Narrow Band Imaging (abbreviated as NBI) mode. The NBI technique can improve the screening rate of early cancer to a certain extent and is beneficial for biopsies. However, due to the body's rejection response, gastroscopy causes significant psychological and physical discomfort to patients.

In recent years, the advent of capsule endoscopes, especially magnetically controlled capsule endoscopes, has provided patients with a comfortable and friendly experience. In terms of WLI screening accuracy, they have met the standards of conventional gastroscopy. However, current capsule endoscopes do not possess NBI imaging capabilities and cannot highlight the mucosal epithelium and submucosal vessels.

### SUMMARY OF THE INVENTION

In order to technically solve the above problems of the prior art, the main object of the present invention is to provide a capsule endoscope imaging device that can clearly display the mucosal epithelium and submucosal vessels of a subject to be tested.

To achieve the above object, the present invention discloses a capsule endoscope imaging device, comprising:
first lamp bodies, which emit white light;
second lamp bodies, which emit light with a central wavelength of 510 nm-540 nm;
third lamp bodies, which emit light with a central wavelength of 400 nm-420 nm; a first control circuit, which is connected to the first lamp bodies, for adjusting exposure time or voltage of the first lamp bodies;
a second control circuit, which is connected to the second lamp bodies, for adjusting exposure time or voltage of the second lamp bodies;
a third control circuit, which is connected to the third lamp bodies, for adjusting exposure time or voltage of the third lamp bodies.

Preferably, before the second control circuit adjusts the exposure time or voltage of the second lamp bodies and the third control circuit adjusts the exposure time or voltage of the third lamp bodies, a ratio of luminous energy of the third lamp bodies to luminous energy of the second lamp bodies is K₁, and after the second control circuit adjusts the exposure time or voltage of the second lamp bodies and the third control circuit adjusts the exposure time or voltage of the third lamp bodies, the ratio of the luminous energy of the third lamp bodies to the luminous energy of the second lamp bodies is K₂, where K₁=K₂.

Preferably, before the second control circuit adjusts the exposure time or voltage of the second lamp bodies and the third control circuit adjusts the exposure time or voltage of the third lamp bodies, the ratio of luminous energy of the third lamp bodies to luminous energy of the second lamp bodies is K₁, and after the second control circuit adjusts the exposure time or voltage of the second lamp bodies and the third control circuit adjusts the exposure time or voltage of the third lamp bodies, the ratio of the luminous energy of the third lamp bodies to the luminous energy of the second lamp bodies is K₂, where K₁≠K₂.

Preferably, the capsule endoscope imaging device further comprises a mounting board, with the first lamp bodies, the second lamp bodies and the third lamp bodies distributed on the mounting board along its circumferential direction.

Preferably, at least two of each of the first lamp bodies, the second lamp bodies, and the third lamp bodies are provided, and the first lamp bodies, the second lamp bodies, and the third lamp bodies are distributed alternately on the mounting board.

Preferably, at least two first lamp bodies are provided, and evenly distributed on the mounting board, with the second lamp bodies and the third lamp bodies also evenly distributed on the mounting board.

Preferably, the first lamp bodies, the second lamp bodies, and the third lamp bodies are LED lights.

Accordingly, the present invention further provides a capsule endoscope, which comprises the said capsule endoscope imaging device.

Accordingly, the present invention further provides an imaging method for the capsule endoscope imaging device, comprising:
pre-setting a brightness value Iᵣ of an image under ideal imaging conditions; acquiring a brightness value I₀ of an image captured by a camera of the capsule endoscope;
comparing the brightness value I₀ with the brightness value Iᵣ, and adjusting the exposure time or voltage of one or more of the first lamp bodies, the second lamp bodies and the third lamp bodies according to the comparison result, to adjust the imaging effect of the first lamp bodies, the second lamp bodies, or the third lamp bodies.

Preferably, when adjusting the imaging effect of the first lamp bodies:
pre-setting a brightness value Iᵣ₁ of a white light image under ideal imaging conditions;
acquiring a brightness value I₀₁ of a white light image captured by the camera;
comparing the brightness value I₀₁ with the brightness value Iᵣ₁, increasing exposure time T_{w1} or voltage V_{w1} of the first lamp bodies when I₀₁ is less than Iᵣ₁; and
decreasing the exposure time T_{w1} or voltage V_{w1} when I₀₁ is greater than Iᵣ₁.

Preferably, in the step of increasing or decreasing the exposure time T_{w1} or voltage V_{w1} of the first lamp bodies,
calculating an initial luminous energy W_{W01} of the first lamp bodies based on an initial voltage V_{W01} and an initial exposure time t_{W01} of the first lamp bodies;
calculating luminous energy W_{Ww1} of the first lamp bodies based on the exposure time T_{w1} or voltage V_{w1} of the first lamp bodies after increase or decrease;
increasing the exposure time T_{w1} or voltage V_{w1} of the first lamp bodies by a control circuit when the brightness value I₀₁ is less than the brightness value Iᵣ₁, to make W_{Ww1}>W_{W01}; and
decreasing the exposure time T_{w1} or voltage V_{w1} of the first lamp bodies by the control circuit when the brightness value I₀₁ is greater than the brightness value Iᵣ₁, to make W_{Ww1}<W_{W01}.

Preferably, when adjusting the imaging effect of the second lamp bodies and the third lamp bodies, pre-setting a brightness value Iᵣ₂ of a narrow-band light image under ideal imaging conditions;
acquiring a brightness value I₀₂ of a narrow-band light image captured by the camera;
comparing the brightness value I₀₂ with the brightness value Iᵣ₂, increasing exposure time T_{w2} and/or voltage V_{w2} of the second lamp bodies and/or the third lamp bodies when I₀₂ is less than Iᵣ₂; and
decreasing the exposure time T_{w2} and/or voltage V_{w2} of the second lamp bodies and/or the third lamp bodies when I₀₂ is greater than Iᵣ₂.

Preferably, before adjusting the exposure time T_{w2} and/or voltage V_{w2} of the second lamp bodies and/or the third lamp bodies, a ratio of luminous energy of the third lamp bodies to luminous energy of the second lamp bodies is K₁, and after adjusting the exposure time T_{w2} and/or voltage V_{w2} of the second lamp bodies and/or the third lamp bodies, the ratio of the luminous energy of the third lamp bodies to the luminous energy of the second lamp bodies is K₂, where K₁=K₂.

Preferably, before adjusting the exposure time T_{w2} and/or voltage V_{w2} of the second lamp bodies and/or the third lamp bodies, a ratio of luminous energy of the third lamp bodies to luminous energy of the second lamp bodies is K₁, and after adjusting the exposure time T_{w2} and/or voltage V_{w2} of the second lamp bodies and/or the third lamp bodies, the ratio of the luminous energy of the third lamp bodies to the luminous energy of the second lamp bodies is K₂, where K₁≠K₂.

Preferably, the step of increasing or decreasing the exposure time T_{w2} or voltage V_{w2} of the second lamp bodies and/or the third lamp bodies comprises:
calculating an initial luminous energy W_{W02} of the second lamp bodies based on an initial voltage V_{W02} and an initial exposure time t_{W02} of the second lamp bodies;
calculating an initial luminous energy W_{W03} of the third lamp bodies based on an initial voltage V_{W03} and an initial exposure time t_{W03} of the third lamp bodies;
increasing or decreasing the exposure time T_{w2} and/or voltage V_{w2} of the second lamp bodies and the third lamp bodies, and calculating luminous energy W_{W2} of the second lamp bodies and luminous energy W_{W3} of the third lamp bodies;
making W_{W2}>W_{W02} and W_{W3}>W_{W03} when the brightness value I₀₂ is less than the brightness value Iᵣ₂;
making W_{W2}<W_{W02} and W_{W3}<W_{W03} when the brightness value I₀₂ is greater than the brightness value Iᵣ₂.

Compared to the prior art, the capsule endoscope imaging device of the present invention comprises first lamp bodies, second lamp bodies, third lamp bodies, a first control circuit, a second control circuit, and a third control circuit. The first control circuit controls the first lamp bodies to emit white light and adjusts its exposure time or voltage to illuminate the mucosa of the subject to be tested with white light and use the reflected white light for imaging, thereby observing the mucosal tissue of the subject to be tested. The second control circuit and third control circuit control the second lamp bodies and third lamp bodies to emit light with different central wavelengths and adjust their exposure time or voltage, making the narrow-band light source composed of the second lamp bodies and third lamp bodies illuminate the mucosa of the subject to be tested and use the reflected narrow-band light for imaging, thereby focusing on observing the mucosal epithelium and submucosal vessels. This achieves switchable white light and narrow-band light illumination within the capsule endoscope, enabling close-range NBI observation of suspected lesion areas. In addition, this endoscope imaging device with NBI imaging capabilities can be used for diagnosing mucosal microvascular lesions, such as early cancer in the middle and lower pharynx, esophageal intraepithelial cancer, early cervical cancer, early colon cancer, etc. These diseases typically cause increased vascularity in the lesion area and changes in the surface structure of the mucosa. Narrow-band imaging endoscopes can timely observe these pathological structures, providing assistance for the early diagnosis and treatment of these diseases.

### BRIEF DESCRIPTION OF THE DRAWINGS

FIG. 1 shows a distribution of first lamp bodies, second lamp bodies and third lamp bodies according to an embodiment of the present invention.

FIGS. 2-8 show distributions of the first lamp bodies, the second lamp bodies and the third lamp bodies according to other embodiments of the present invention.

FIG. 9 shows a control circuit of the first lamp bodies according to an embodiment of the present invention.

FIG. 10 shows a control circuit of the second lamp bodies according to an embodiment of the present invention.

FIG. 11 shows a control circuit of the third lamp bodies according to an embodiment of the present invention.

FIG. 12 shows a flowchart of an imaging method according to an embodiment of the present invention.

FIG. 13 shows a circuit diagram for switching between the second lamp bodies and the third lamp bodies according to an embodiment of the present invention.

FIG. 14 shows a spectral power diagram of lamp bodies LG1 according to an embodiment of the present invention.

FIG. 15 shows a spectral power diagram of lamp bodies LB1 according to an embodiment of the present invention.

FIGS. 16-18 show narrow-band spectrum diagrams of the lamp bodies LG1 and the lamp bodies LB1 according to the embodiments of the present invention.

FIG. 19 shows a schematic view of CMOS sensor processing narrow-band light images according to the embodiments of the present invention.

An element in the drawings is: 1 Mounting board.

### DETAILED DESCRIPTION

In order to make the objects, technical solutions, and advantages of the present invention more understandable, the present invention will be further described in detail below with reference to the accompanying drawings and embodiments. It will be appreciated by those skilled in the art that the following discussion is for demonstration purposes, and should not be interpreted as a limitation. Other variances within the scope of this disclosure are also applicable.

The present invention discloses a capsule endoscope imaging device. The capsule endoscope imaging device comprises a mounting board, first lamp bodies, second lamp bodies and third lamp bodies. The first lamp bodies, the second lamp bodies and the third lamp bodies are arranged on the mounting board. The first lamp bodies emit white light to illuminate the mucosa of a subject to be tested and capture images through a camera of a capsule endoscope. The second and third lamp bodies form a narrow-band light source to illuminate the mucosa of the subject to be tested and capture images through the camera of the capsule endoscope, thus facilitating observation of the subject to be tested.

The present invention uses the white light emitted by the first lamp bodies to illuminate the mucosa of the subject to be tested, captures images using the reflected white light to observe the mucosal tissue, and uses the narrow-band light source to illuminate the mucosa of the subject to be tested, captures images using the reflected narrow-band light to observe the mucosal epithelium and submucosal vessels of the subject to be tested. The method achieves switching between white light illumination and narrow-band light illumination in the capsule endoscope. Initial observation is conducted using white light illumination, and suspected lesion areas can be observed by switching to close-up NBI observation, enhancing the completeness of the observation. In the embodiment, the subject to be tested may be the digestive tract of a human or animal body.

In an embodiment of the present invention, the first lamp bodies, the second lamp bodies, and the third lamp bodies are distributed on the mounting board 1 along its circumferential direction.

In an embodiment of the present invention, at least two of each of the first lamp bodies, the second lamp bodies, and the third lamp bodies are provided, and the first lamp bodies, the second lamp bodies, and the third lamp bodies are distributed alternately on the mounting board 1.

In an embodiment of the present invention, at least two first lamp bodies are provided, and evenly distributed on the mounting board 1, with the second lamp bodies and the third lamp bodies also evenly distributed on the mounting board 1.

Specifically, as shown in FIG. 1, the first lamp bodies may comprise four lamp bodies LW1, the second lamp bodies may comprise two lamp bodies LG1, and the third lamp bodies may comprise two lamp bodies LB1. The lamp bodies LW1, the lamp bodies LG1 and the lamp bodies LB1 all distributed alternately and evenly on the mounting board 1 along its circumferential direction.

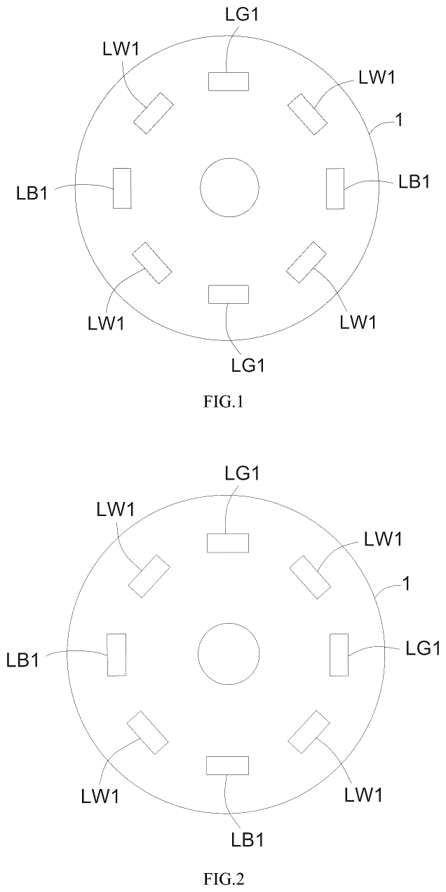

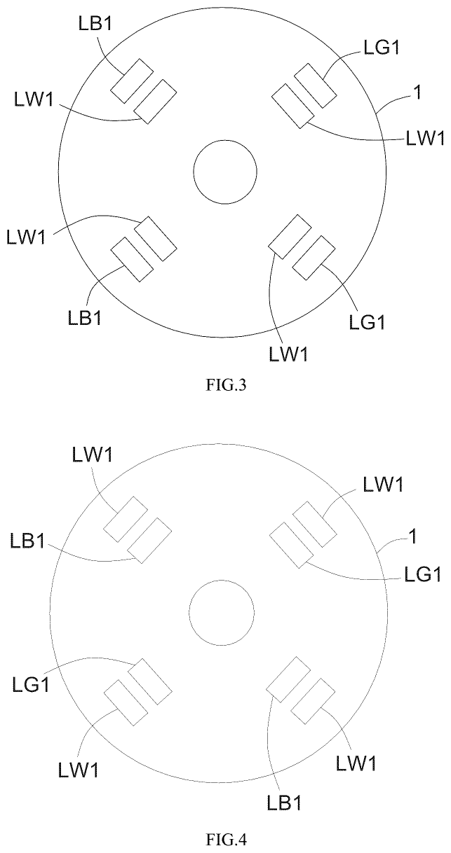

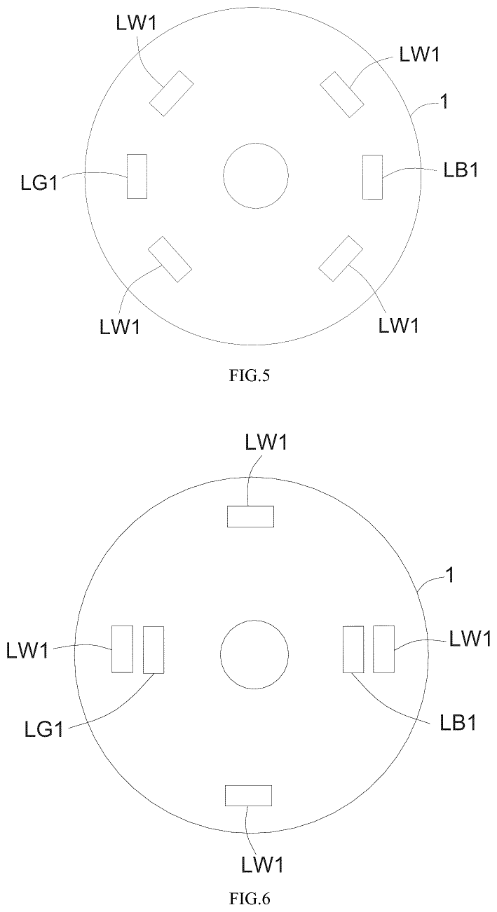

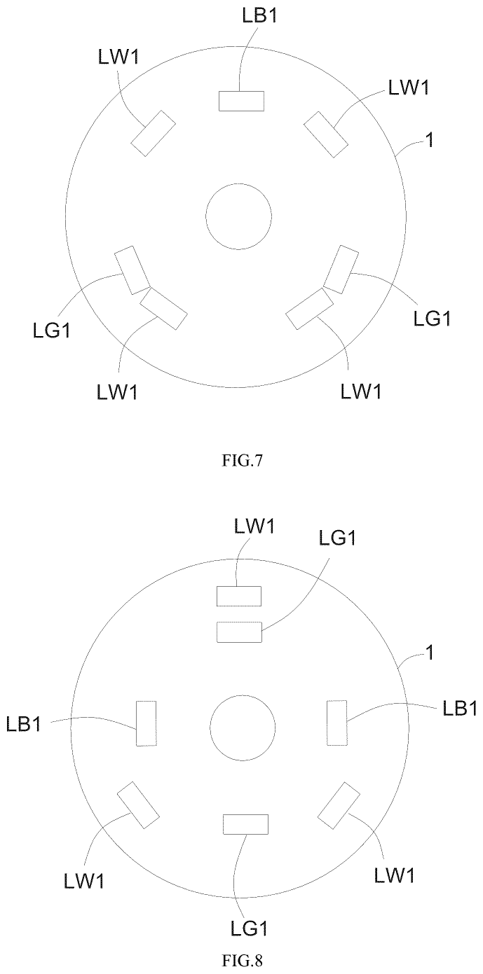

In the embodiment, the first lamp bodies comprise four lamp bodies LW1, the second lamp bodies comprise two lamp bodies LG1, and the third lamp bodies comprise two lamp bodies LB1, totaling 8 lamp bodies, all of which are distributed alternately and evenly on the mounting board 1 along its circumferential direction. It can be understood that in other embodiments, the number of lamp bodies LW1 is not limited to four and may be less than or more than four; the number of lamp bodies LG1 or lamp bodies LB1 is also not limited to two and may be less or more than two. Additionally, the number of lamp bodies LW1 does not have to equal the total number of the lamp bodies LG1 and lamp bodies LB1; the number of lamp bodies LW1 may be less or more than the total number of the lamp bodies LG1 and lamp bodies LB1. Moreover, the lamp bodies LW1, lamp bodies LG1, and lamp bodies LB1 are not limited to being distributed alternately and evenly on the mounting board along its circumferential direction. It is possible that the lamp bodies LW1 may be distributed on the mounting board along its circumferential direction while the lamp bodies LG1 and lamp bodies LB1 may be distributed on the mounting board closer to or farther from the center compared to the lamp bodies LW1. Preferably, the lamp bodies LW1 are evenly distributed on the mounting board along its circumferential direction, and the lamp bodies LB1 and lamp bodies LG1 are also evenly distributed on the mounting board along its circumferential direction. For example, when there are three lamp bodies LW1, they are arranged at 120 degrees around the center of the mounting board. For another example, when the total number of the lamp bodies LB1 and lamp bodies LG1 is four, they are arranged at 90 degrees around the center of the mounting board. Specifically, as shown in FIGS. 2, 3, 5, 6 and 7, the four lamp bodies LW1 are evenly spaced and distributed along the circumferential direction of the mounting board 1, the lamp bodies LB1 and lamp bodies LG1 are distributed along the radial direction of the mounting board 1 closer to or farther from the center compared to the lamp bodies LW1, and the total number of LB1 and lamp bodies LG1 may be less than or equal to the number of lamp bodies LW1. Referring to FIG. 4, the lamp bodies LB1 and lamp bodies LG1 may be symmetrically distributed about the center of the mounting board 1, and the lamp bodies LW1 may be symmetrically arranged about the line connecting the lamp bodies LB1 and lamp bodies LG1.

Specifically, the central wavelength of the light emitted by the lamp bodies LG1 is 510 nm-540 nm. Preferably, the central wavelength of the light emitted by the lamp bodies LG1 is 525 nm, with a bandwidth of 480 nm-580 nm. The central wavelength of the light emitted by the lamp bodies LB1 is 400 nm-420 nm. Preferably, the central wavelength of the light emitted by the lamp bodies LB1 is 415 nm, with a bandwidth of 390 nm-450 nm.

In the embodiment, the lamp bodies LW1, lamp bodies LG1, and lamp bodies LB1 are all light emitting diode (abbreviated as LED) lights. The lamp bodies LW1, lamp bodies LG1, and lamp bodies LB1 in the present invention are all LED lights, which can effectively reduce the space occupied by the lamp bodies in design, thereby allowing them to be arranged inside the capsule endoscope. Additionally, using LED lights as the light source is cost-effective. It can be understood that in other embodiments, the lamp bodies LW1, lamp bodies LG1, and lamp bodies LB1 may also be other types of lights, such as small lasers.

Traditional electronic endoscopes use broad-band white light sources such as xenon lamps or tungsten halogen lamps for illumination, allowing all wavelengths of visible light to pass through the filter. Narrow-band imaging endoscopes have narrow-band filters disposed after the white light sources to filter out the broadband white light, leaving only a limited number of narrow-band spectra with central wavelengths of 605 nm, 540 nm, and 415 nm, which are then transmitted to the target surface. Due to the different penetration depths of narrow-band light waves into the mucosa and the strong absorption of blue light and green light by blood (especially hemoglobin) in the mucosa, using light waves that are difficult to scatter and can be absorbed by blood increases the contrast and clarity of the mucosal epithelium and submucosal vessels.

By decomposing and analyzing the captured images according to different spectra, images at different mucosal depths can be obtained for better observation.

The present invention redesigns the illumination light source of the capsule endoscope to incorporate both white light illumination (abbreviated as WLI) and narrow-band imaging (abbreviated as NBI) functions. Switching between white light imaging and narrow-band imaging is realized through circuit control to obtain white light images and narrow-band images. Thereby, the narrow-band imaging function can highlight information about the mucosal epithelium and submucosal blood vessels.

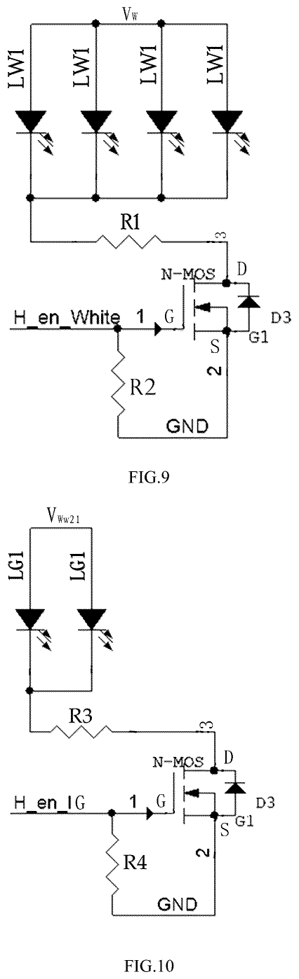

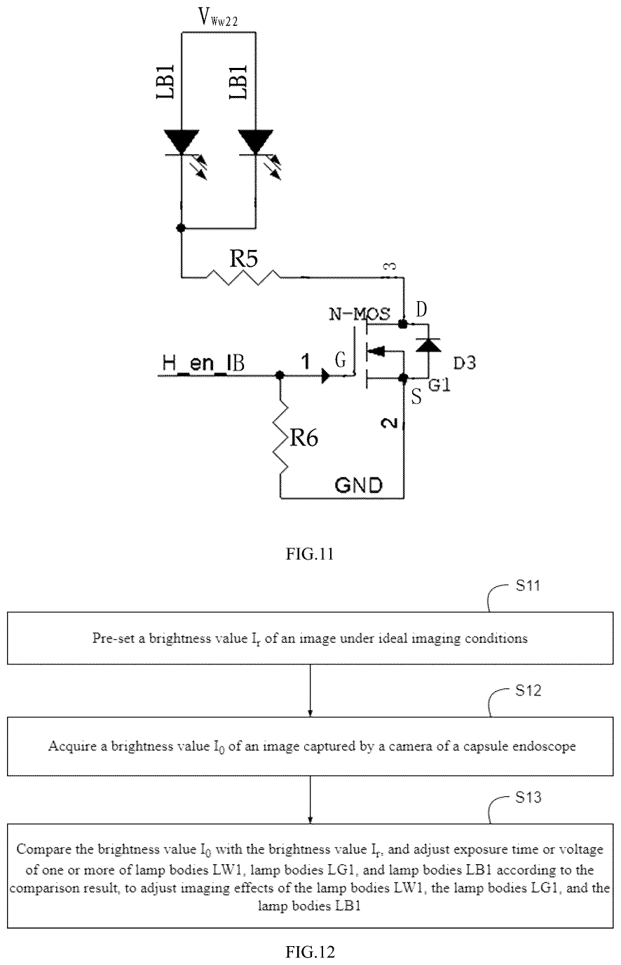

As shown in FIGS. 9, 10 and 11, the capsule endoscope imaging device of the present invention further comprises a first control circuit, a second control circuit, and a third control circuit. The first control circuit comprises a control circuit H_en_White and four lamp bodies LW1 connected in parallel with each other. The control circuit H_en_White is connected to the four lamp bodies LW1. The second control circuit comprises a control circuit H_en_IG and two lamp bodies LG1 connected in parallel with each other. The control circuit H_en_IG is connected to the two lamp bodies LG1. The third control circuit comprises a control circuit H_en_IB and two lamp bodies LB1 connected in parallel with each other. The control circuit H_en_IB is connected to the two lamp bodies LB1.

The capsule endoscope imaging device of the present invention further comprises a switching circuit, which is used to switch between the first control circuit, the second control circuit, and the third control circuit. Specifically, the switching circuit controls the output levels of the control circuit H_en_White, control circuit H_en_IG, and control circuit H_en_IB to switch between the control circuits. When the control circuit H_en_White outputs a high level, the four lamp bodies LW1 light up; when the control circuit H_en_IG outputs a high level, the two lamp bodies LG1 light up; when the control circuit H_en_IB outputs a high level, the two lamp bodies LB1 light up. The levels of the control circuit H_en_IB and control circuit H_en_IG remain consistent, high or low simultaneously. The level of the control circuit H_en_White alternates with the control circuit H_en_IG and control circuitH_en_IB, that is: when the control circuit H_en_White outputs a high level, the control circuit H_en_IG and control circuit H_en_IB output a low level; when the control circuit H_en_White outputs a low level, the control circuit H_en_IG and control circuit H_en_IB simultaneously output a high level, ensuring the sequential switching of the simultaneous lighting of lamp bodies LG1 and lamp bodies LB1 with the lighting of lamp bodies LW1, thus providing the basis for switching between white light source and narrow-band light source for imaging.

As shown in FIG. 12, accordingly, the present invention further discloses an imaging method for the capsule endoscope imaging device. According to the imaging effect of the images captured by a camera of the capsule endoscope, the method calculates through an algorithm to automatically adjust the voltage division and respective exposure times of the lamp bodies LW1, lamp bodies LG1, and lamp bodies LB1 to optimize the imaging effect. The specific adjustment process comprises:
step S11, pre-setting the brightness value Iᵣ of the image under ideal imaging conditions;
step S12, acquiring the brightness value I₀ of the image captured by the camera;
step S13, comparing the brightness value I₀ with the brightness value Iᵣ, and adjusting the exposure time or voltage of one or more of the lamp bodies LW1, the lamp bodies LG1, and the lamp bodies LB1 according to the comparison result, to adjust the imaging effects of the lamp bodies LW1, the lamp bodies LG1, and the lamp bodies LB1.

For ease of description, in the following embodiments, T_{w1} is a general description of the exposure time for the first lamp bodies, including t_{W01} and t_{Ww1}.

Where, t_{W01} is the specific initial exposure time of the first lamp bodies, and t_{Ww1} is the specific exposure time of the first lamp bodies after adjustment. T_{w2} is a general description of the exposure time for the second lamp bodies, including t_{W02} and t_{Ww21}. Where, t_{w02} is the specific initial exposure time of the second lamp bodies, and t_{Ww21} is the specific exposure time of the second lamp bodies after adjustment. T_{w3} is a general description of the exposure time for the third lamp bodies, including t_{w03} and t_{Ww22}. Where, t_{W03} is the specific initial exposure time of the third lamp bodies, and t_{Ww22} is the specific exposure time of the third lamp bodies after adjustment.

When adjusting the imaging effect of the first lamp bodies, the specific operation process is:
when the switching circuit switches to drive the first lamp bodies to work, the control circuit applies an initial voltage V_{W01} to the lamp bodies LW1 and sets the initial exposure time t_{W01}, and calculates the luminous energy W_{W01} of the lamp bodies LW1 within the single exposure time t_{W01} using the initial voltage V_{W01} and the initial exposure time t_{W01} of the lamp bodies LW1. W_{W01}=P_{W01}*t_{W01}, where P_{W01} refers to the power of the lamp bodies LW1, and is the sum of the power of all lamp bodies LW1 at the voltage V_{W01}.

At this point, the CMOS sensor in the camera captures a white light image. By comparing the brightness value I₀₁ of the white light image with the brightness value Iᵣ₁ of the white light image under ideal imaging conditions, the luminous energy of the first light source is adjusted when capturing the next frame of image to adjust the brightness value of the next frame of image, where the luminous energy of the first light source is positively correlated with the brightness value of the image taken under the illumination of the first light source. When I₀₁<Iᵣ₁, the control circuit increases the exposure time T_{w1} or the voltage V_{W1} of the lamp bodies LW1, that is, makes the luminous energy W_{Ww1} of the lamp bodies LW1 greater than W_{W01}, W_{Ww1}>W_{W01}, so that the brightness of the next frame of image is increased.

W_{Ww1}=P_{Ww1}*t_{Ww1}, where P_{Ww1} is the sum of the power of all lamp bodies LW1 when the adjusted voltage is V_{W1}. On the contrary, when I₀₁>Iᵣ₁, the control circuit decreases the exposure time T_{w1} or voltage V_{W1} of the lamp bodies LW1, so that W_{Ww1}<W_{W01}, thereby reducing the luminous energy of the first lamp bodies when capturing the next frame of the image, and decreasing the brightness of the next frame of the image. Through the above adjustment, the brightness of the white light image can achieve the best effect.

When adjusting the imaging effect of the lamp bodies LG1 and the lamp bodies LB1, the specific operation process is:
when the control circuit switches to drive the lamp bodies LG1 and LB1 to work simultaneously, the control circuit applies an initial voltage V_{W02} and an initial exposure time t_{W02} to the lamp bodies LG1, and calculates the luminous energy of the lamp bodies LG1 within the single exposure time t_{W02} using the initial voltage V_{W02} and initial exposure time t_{W02} of the lamp bodies LG1: W_{w02}=P_{W02}*t_{W02}, where P_{W02} is the sum of the power of all lamp bodies LG1 (i.e., the second lamp bodies) when the voltage is V_{W02}.

Additionally, the control circuit applies an initial voltage V_{W03} and an initial exposure time t_{W03} to the lamp bodies LB1, and calculates the luminous energy of the lamp bodies LB1 within the single exposure time t_{W03} using the initial voltage V_{W03} and the initial exposure time t_{W03}: W_{W03}=P_{W03}*t_{W03}, where P_{W03} is the sum of the power of all lamp bodies LB1 (i.e., the third lamp bodies) when the voltage is V_{W03}.

At this point, the CMOS sensor in the camera captures a narrow-band light image. By comparing the brightness value I₀₂ of the narrow-band light image with the brightness value Iᵣ₂ of the narrow-band light image under ideal imaging conditions, the luminous energy of the second light source is adjusted when capturing the next frame of image to adjust the brightness value of the next frame of image, where, the luminous energy of the lamp bodies LG1 and lamp bodies LB1 is positively correlated with the brightness value of the image taken under the illumination of the lamp bodies LG1 and lamp bodies LB1. The adjustment can be achieved by adjusting the exposure time T_{w2} and/or the voltage V_{w2} of the lamp bodies LG1 and/or the lamp bodies LB1.

When I₀₂<Iᵣ₂, the exposure time T_{w2} and/or voltage V_{W2} of the lamp bodies LG1 and/or lamp bodies LB1 are increased, so that W_{W2}>W_{W02}, W_{W3}>W_{W03}. Where, W_{W2}=P_{Ww21}*t_{Ww21}, W_{W3}=P_{Ww22}*t_{Ww22}, where P_{Ww21} is the sum of the light intensity power of all lamp bodies LG1 after voltage adjustment, t_{Ww21} is the exposure time of the lamp bodies LG1 after adjustment, and W_{w2} is the total luminous energy of all lamp bodies LG1 after adjustment; P_{Ww22} is the sum of the light intensity power of all lamp bodies LB1 after voltage adjustment, t_{ww22} is the exposure time of the lamp bodies LB1 after adjustment, and W_{W3} is the total luminous energy of all lamp bodies LB1 after adjustment.

On the contrary, when I₀₂>Iᵣ₂, the control circuit decreases the exposure time T_{W2} and/or the voltage V_{W2} of the lamp bodies LG1 and/or the lamp bodies LB1, making W_{W2}<W_{W02}, W_{W3}<W_{W03}, thereby reducing the brightness of the image in the next frame. Through the above adjustments, the brightness of the narrow-band light image can achieve the best effect.

The method of processing narrow-band light images with a CMOS sensor is shown in FIG. 19. FIG. 19 shows the synthesis process of an NBI image. Under the illumination of light with a wavelength of 415 nm in the narrow-band spectrum, the image brightness formed is used as the G and B (i.e., the brightness values of the green channel and blue channel) in the final synthesized color image. Under the illumination of light with a wavelength of 540 nm in the narrow-band spectrum, the image brightness formed is used as the R (i.e., the brightness value of the red channel) in the final synthesized color image. Finally, R, G, and B are synthesized into the displayed color image.

Further, the R, G, B channels in the reconstructed NBI image are synthesized from the obtained original g channel and b channel information, where the NBI_R channel information is given by the g channel information, and the NBI_G channel and NBI_B channel information is given by the b channel information, which is expressed in a mathematical formula:

$$[ \begin{matrix}NBI\_R \\ NBI\_G \\ NBI\_B\end{matrix} ] = [ \begin{matrix}0 & m 1 & 0 \\ 0 & 0 & m 2 \\ 0 & 0 & m 3\end{matrix} ] \times [ \begin{matrix}r \\ g \\ b\end{matrix} ] .$$

Where, NBI_R is the brightness value of the R channel in the NBI color image, NBI_G is the brightness value of the G channel in the NBI color image, and NBI_B is the brightness value of the B channel in the NBI color image; m1, m2, and m3 are parameters determined based on the original image values to achieve optimal NBI imaging effects; r is the brightness value of the r channel in the original NBI image, g is the brightness value of the g channel in the original NBI image, and b is the brightness value of the b channel in the original NBI image.

Preferably, the imaging method provided by the present invention can also adjust the ratio of the luminous energy of the lamp bodies LG1 and/or the lamp bodies LB1 to achieve different imaging effects. In one embodiment, the ratio of the luminous energy before and after the adjustment of the lamp bodies LG1 and the lamp bodies LG2 is respectively W_{W03}/W_{w02}=K₁ and W_{W3}/W_{W2}=K₂. When enhancing or reducing the luminous energy, it is possible to achieve proportional adjustment of the luminous energy, that is, K1=K2. In other embodiments, the ratio of the luminous energy during the two image acquisition processes can also be adjusted to be unequal, i.e., K1≠K2.

More specifically, in the above-mentioned adjustment process, it is possible to adjust only the voltage V_{Ww21} of the lamp bodies LG1 and/or the voltage V_{Ww22} of the lamp bodies LB1, or adjust only the exposure time of the lamp bodies LG1 and/or the exposure time of the lamp bodies LB1, or adjust both the exposure time and voltage of the lamp bodies LG1 and lamp bodies LG2 simultaneously. It should be noted that in the adjustment process mentioned above, the voltage V_{Ww21} of the lamp bodies LG1 and the voltage V_{Ww22} of the lamp bodies LB1 change in the same direction, that is, when the voltage V_{Ww21} of the lamp bodies LG1 increases, the voltage V_{Ww22} of the lamp bodies LB1 also increases, and when the voltage V_{Ww21} of the lamp bodies LG1 decreases, the voltage V_{Ww22} of the lamp bodies LB1 also decreases.

For example: in one embodiment, it is possible to adjust only the voltage V_{Ww21} of the lamp bodies LG1 and the voltage V_{Ww22} of the lamp bodies LB1; in one embodiment, it is possible to adjust only the exposure time of the lamp bodies LG1 and the exposure time of the lamp bodies LB1; in one embodiment, it is possible to adjust only the voltage V_{Ww21} of the lamp bodies LG1 or the voltage V_{Ww22} of the lamp bodies LB1; in one embodiment, it is possible to adjust only the exposure time of the lamp bodies LG1 or the exposure time of the lamp bodies LB1. In other embodiments, the exposure time and voltage of both lamp bodies LG1 and lamp bodies LG2 can be adjusted simultaneously. It is also possible to adjust only the exposure time of both lamp bodies LG1 and lamp bodies LG2 simultaneously, or to adjust only the voltage of both lamp bodies LG1 and lamp bodies LG2 simultaneously. From the adjustment results, it can be seen that the above adjustment method can adjust the proportional or non-proportional lighting of the lamp bodies LG1 and/or the lamp bodies LB1.

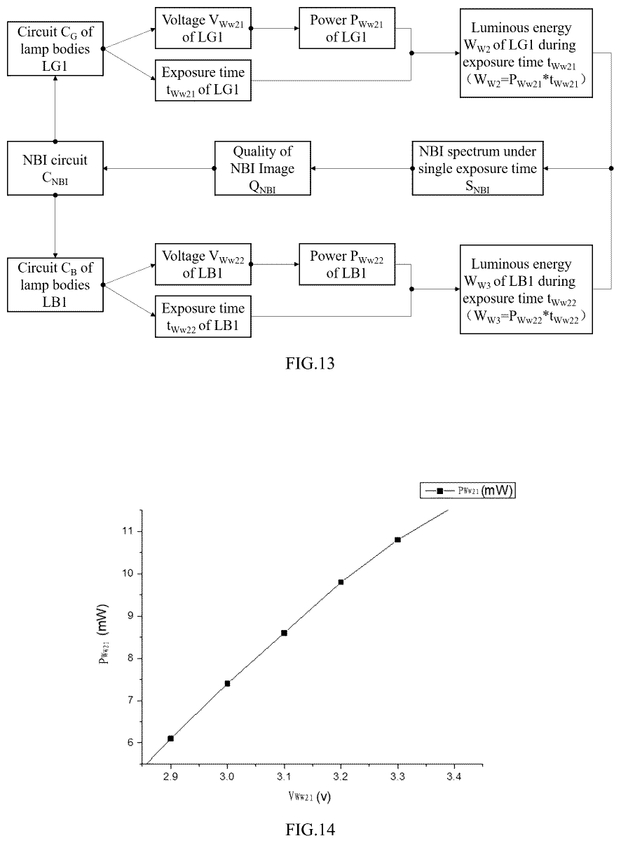

As shown in FIG. 13, in the embodiment of the present invention, the lamp bodies LG1 and the lamp bodies LB1 work through circuit switching, and the NBI circuit C_{NBI} controls the circuit C_{G} of the lamp bodies LG1 and the circuit C_{B} of the lamp bodies LB1. The circuit C_{G} and circuit C_{B} control the voltage V_{Ww21}, voltage V_{Ww22} and the single exposure time t_{Ww21}, single time exposure time t_{Ww22} of the lamp bodies LG1 and lamp bodies LB1, respectively. Where, the NBI circuit C_{NBI} comprises the switching circuit, the circuit C_{G} comprises the control circuit H_en_IG, and the circuit C_{B} comprises the control circuit H_en_IB. Taking the lamp bodies LG1 as an example, the circuit C_{G} controls the voltage V_{Ww21} and the exposure time t_{Ww21} of the lamp bodies LG1. The luminous power P_{Ww21} of the lamp bodies LG1 varies with different driving voltages V_{Ww21}. The higher the driving voltage V_{Ww21}, the higher the narrow-band spectrum power P_{Ww21} of the lamp bodies LG1, as shown in FIG. 14.

The circuit C_{G} simultaneously controls the exposure time t_{Ww21} of the lamp bodies LG1, and the luminous energy W_{W2} of the narrow-band light of the lamp bodies LG1 under a single exposure: W_{W2}=P_{Ww21}*t_{Ww21}.

The longer the single exposure time, the greater the narrow-band energy.

Similarly, for the spectral characteristics of the lamp bodies LB1 controlled by the circuit C_{B}, its parameters are the same as those controlled by the lamp bodies LG1, as shown in FIG. 15.

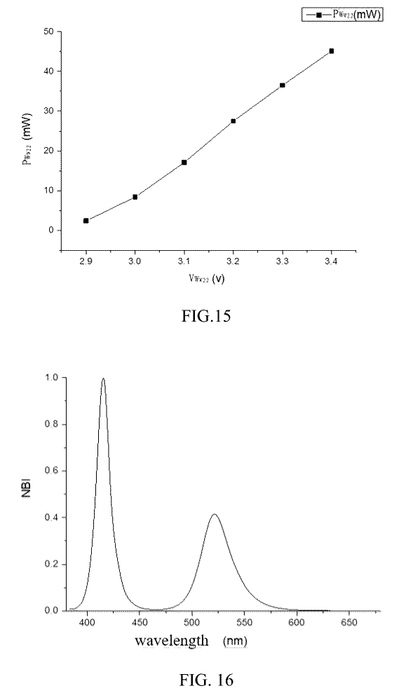

The power of the narrow-band spectrum varies under different driving voltages V_{Ww22}, as shown in FIG. 16.

The circuit C_{B} simultaneously controls the exposure time t_{Ww22} of the lamp bodies LB1, and the luminous energy W_{W3} of the narrow-band light of the lamp bodies LB1 under a single exposure: W_{W3}=P_{Ww22}*t_{Ww22}.

The final effect of the circuit control is that, when W_{W3}/W_{W2}=2, the energy of the narrow-band light is adjusted based on the image brightness of the previous frame of narrow-band light imaging to achieve the optimal narrow-band imaging effect. That is, under the premise of satisfying W_{W3}/W_{W2}=2 for each exposure, when the narrow-band image of the previous frame is relatively dark, the circuit C_{NBI} controls the lamp bodies LG1 and the lamp bodies LB1 to increase the luminous energy of the narrow-band light for this exposure, thereby increasing the image brightness. Similarly, when the narrow-band image of the previous frame is brighter, the circuit C_{NBI} controls the lamp bodies LG1 and the lamp bodies LB1 to reduce the narrow-band light energy for this exposure, thereby reducing the image brightness.

The control of the lamp bodies LG1 and the lamp bodies LB1 by the second control circuit and the third control circuit can be adjusted by adjusting the voltage of each lamp body to regulate the luminous power, as well as controlling different exposure times t_{Ww21} and t_{Ww22}, or simultaneously controlling the voltages V_{Ww21}, V_{Ww22} and the exposure times t_{Ww21}, t_{Ww22}, ultimately achieving the best imaging effect.

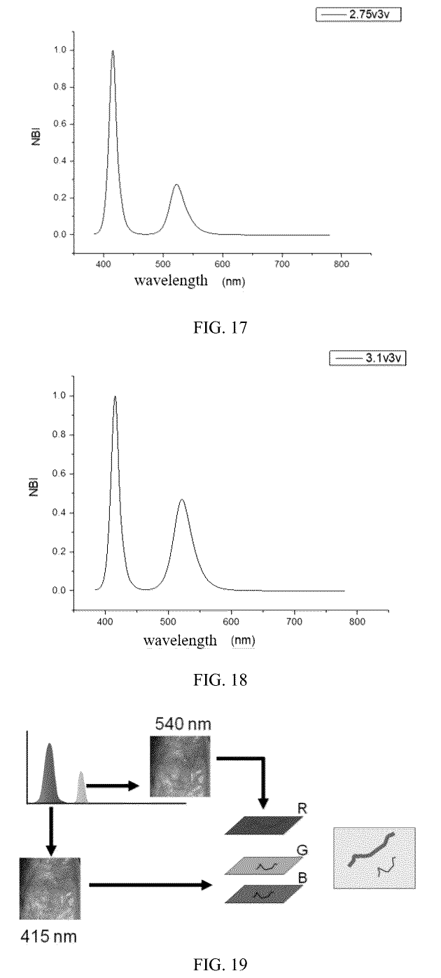

For example, in a certain exposure, the voltage and exposure time of the lamp bodies LG1 are V_{Ww21}=3v and t_{Ww21}=2.8 ms, and the sum of power of all lamp bodies LG1 at a voltage of 3V is P_{Ww21}=7.5 mW. The voltage and exposure time of the lamp bodies LB1 are V_{Ww22}=3v and t_{Ww22}=5 ms, and the sum of power of all lamp bodies LB1 at a voltage of 3V is P_{Ww22}=8.4 mW. It is obtained that W_{W2}=21uJ, W_{W3}=42uJ, W_{W3}/W_{W2}=2. Its narrow-band light is shown in FIG. 17. In the final narrow-band spectrum, the exposure time of the lamp bodies LB1 is longer than that of the lamp bodies LG1, ensuring that the single exposure W_{W3}/W_{W2}=2.

Similarly, by controlling the voltage, for example, during a certain exposure, the voltage and exposure time of the lamp bodies LG1 are V_{Ww21}=2.75v and t_{Ww21}=5 ms, and the sum of power of all lamp bodies LG1 at a voltage of 2.75V is P_{Ww21}=4.2 mW. The voltage and exposure time of the lamp bodies LB1 are V_{Ww22}=3v and t_{Ww22}=5 ms, and the sum of power of all lamps LB1 at a voltage of 3V is P_{Ww22}=8.4 mW. It is obtained that W_{W2}=21uJ, W_{W3}=42uJ, W_{W3}/W_{W2}=2. The narrow-band spectrum diagram of the lamp bodies LG1 and the lamp bodies LB1 is shown in FIG. 18.

Similarly, the circuit can also simultaneously control the voltage and exposure time of the lamp bodies LG1 and lamp bodies LB1, ensuring the best narrow-band light imaging effect. For example, in a certain exposure, the voltage and exposure time of the lamp bodies LG1 are V_{Ww21}=3.1v and t_{Ww21}=2.4 ms, and the sum of power of all lamp bodies LG1 at a voltage of 3.1V is P_{Ww21}=8.75 mW. The voltage and exposure time of the lamp bodies LB1 are V_{Ww22}=3v and t_{Ww22}=5 ms, and the sum of power of all lamp bodies LB1 at a voltage of 3V is P_{Ww22}=8.4 mW. It is obtained that W_{W2}=2uJ, W_{W3}=42uJ, W_{W3}/W_{W2}=2.

The present invention further discloses a capsule endoscope, which comprises the capsule endoscope imaging device as described above.

The foregoing is only preferred specific embodiments of the present invention, but the scope of protection of the present invention is not limited thereto. Any modifications or substitutions that can be readily thought of by any person skilled in the art within the scope of the technology disclosed by the present invention should be covered by the scope of protection of the present invention. Therefore, the scope of protection of the present invention should be subject to the scope of protection of the claims.

1. A capsule endoscope imaging device, comprising:
first lamp bodies, which emit white light;
second lamp bodies, which emit light with a central wavelength of 510 nm-540 nm;
third lamp bodies, which emit light with a central wavelength of 400 nm-420 nm;
a first control circuit, which is connected to the first lamp bodies, for adjusting exposure time or voltage of the first lamp bodies;
a second control circuit, which is connected to the second lamp bodies, for adjusting exposure time or voltage of the second lamp bodies;
a third control circuit, which is connected to the third lamp bodies, for adjusting exposure time or voltage of the third lamp bodies.

2. The capsule endoscope imaging device of claim 1, wherein before the second control circuit adjusts the exposure time or voltage of the second lamp bodies and the third control circuit adjusts the exposure time or voltage of the third lamp bodies, a ratio of luminous energy of the third lamp bodies to luminous energy of the second lamp bodies is K₁, and after the second control circuit adjusts the exposure time or voltage of the second lamp bodies and the third control circuit adjusts the exposure time or voltage of the third lamp bodies, the ratio of the luminous energy of the third lamp bodies to the luminous energy of the second lamp bodies is K₂, wherein K1=K2.

3. The capsule endoscope imaging device of claim 1, wherein before the second control circuit adjusts the exposure time or voltage of the second lamp bodies and the third control circuit adjusts the exposure time or voltage of the third lamp bodies, the ratio of luminous energy of the third lamp bodies to luminous energy of the second lamp bodies is K₁, and after the second control circuit adjusts the exposure time or voltage of the second lamp bodies and the third control circuit adjusts the exposure time or voltage of the third lamp bodies, the ratio of the luminous energy of the third lamp bodies to the luminous energy of the second lamp bodies is K₂, wherein K₁≠K₂.

4. The capsule endoscope imaging device of claim 1, wherein the capsule endoscope imaging device further comprises a mounting board, with the first lamp bodies, the second lamp bodies and the third lamp bodies distributed on the mounting board along its circumferential direction.

5. The capsule endoscope imaging device of claim 4, wherein at least two of each of the first lamp bodies, the second lamp bodies, and the third lamp bodies are provided, and the first lamp bodies, the second lamp bodies, and the third lamp bodies are distributed alternately on the mounting board.

6. The capsule endoscope imaging device of claim 4, wherein at least two first lamp bodies are provided, and evenly distributed on the mounting board, with the second lamp bodies and the third lamp bodies also evenly distributed on the mounting board.

7. The capsule endoscope imaging device of claim 1, wherein the first lamp bodies, the second lamp bodies, and the third lamp bodies are LED lights.

8. A capsule endoscope, wherein the capsule endoscope comprises the capsule endoscope imaging device according to claim 1.

9. An imaging method for the capsule endoscope imaging device according to claim 1, wherein the imaging method comprises:
pre-setting a brightness value Iᵣ of an image under ideal imaging conditions;
acquiring a brightness value I₀ of an image captured by a camera of the capsule endoscope;
comparing the brightness value I₀ with the brightness value Iᵣ, and adjusting the exposure time or voltage of one or more of the first lamp bodies, the second lamp bodies and the third lamp bodies according to the comparison result, to adjust the imaging effect of the first lamp bodies, the second lamp bodies, or the third lamp bodies.

10. The imaging method of claim 9, wherein, when adjusting the imaging effect of the first lamp bodies,
pre-setting a brightness value Iᵣ₁ of a white light image under ideal imaging conditions;
acquiring a brightness value I₀₁ of a white light image captured by the camera;
comparing the brightness value I₀₁ with the brightness value Iᵣ₁, increasing exposure time Twi or voltage V_{w1} of the first lamp bodies when I₀₁ is less than Iᵣ₁; and
decreasing the exposure time T_{w1} or voltage V_{w1} when I₀₁ is greater than Iᵣ₁.

11. The imaging method of claim 10, wherein, in the step of increasing or decreasing the exposure time T_{w1} or voltage V_{w1} of the first lamp bodies,
calculating an initial luminous energy W_{W01} of the first lamp bodies based on an initial voltage V_{W01} and an initial exposure time t_{W01} of the first lamp bodies;
calculating luminous energy W_{Ww1} of the first lamp bodies based on the exposure time T_{w1} or voltage V_{w1} of the first lamp bodies after increase or decrease;
increasing the exposure time T_{w1} or voltage V_{w1} of the first lamp bodies by a control circuit when the brightness value I₀₁ is less than the brightness value Iᵣ₁, to make W_{Ww1}>W_{W01}; and
decreasing the exposure time T_{w1} or voltage V_{w1} of the first lamp bodies by the control circuit when the brightness value I₀₁ is greater than the brightness value Iᵣ₁, to make W_{Ww1}<W_{W01}.

12. The imaging method of claim 9, wherein, when adjusting the imaging effect of the second lamp bodies and the third lamp bodies,
pre-setting a brightness value Iᵣ₂ of a narrow-band light image under ideal imaging conditions;
acquiring a brightness value I₀₂ of a narrow-band light image captured by the camera;
comparing the brightness value I₀₂ with the brightness value Iᵣ₂, increasing exposure time T_{w2} and/or voltage VW₂ of the second lamp bodies and/or the third lamp bodies when I₀₂ is less than Iᵣ₂; and
decreasing the exposure time T_{W2} and/or voltage V_{w2} of the second lamp bodies and/or the third lamp bodies when I₀₂ is greater than Iᵣ₂.

13. The imaging method of claim 12, wherein, before adjusting the exposure time T_{W2} and/or voltage VW₂ of the second lamp bodies and/or the third lamp bodies, a ratio of luminous energy of the third lamp bodies to luminous energy of the second lamp bodies is K₁, and after adjusting the exposure time T_{W2} and/or voltage V_{w2} of the second lamp bodies and/or the third lamp bodies, the ratio of the luminous energy of the third lamp bodies to the luminous energy of the second lamp bodies is K₂, wherein K1=K2.

14. The imaging method of claim 12, wherein, before adjusting the exposure time T_{W2} and/or voltage VW₂ of the second lamp bodies and/or the third lamp bodies, a ratio of luminous energy of the third lamp bodies to luminous energy of the second lamp bodies is K₁, and after adjusting the exposure time T_{W2} and/or voltage V_{w2} of the second lamp bodies and/or the third lamp bodies, the ratio of the luminous energy of the third lamp bodies to the luminous energy of the second lamp bodies is K₂, wherein K₁≠K₂.

15. The imaging method of claim 12, wherein the step of increasing or decreasing the exposure time T_{W2} or voltage V_{w2} of the second lamp bodies and/or the third lamp bodies comprises:
calculating an initial luminous energy W_{W02} of the second lamp bodies based on an initial voltage V_{W02} and an initial exposure time t_{W02} of the second lamp bodies;
calculating an initial luminous energy W_{W03} of the third lamp bodies based on an initial voltage V_{W03} and an initial exposure time t_{W03} of the third lamp bodies;
increasing or decreasing the exposure time T_{W2} and/or voltage V_{w2} of the second lamp bodies and the third lamp bodies, and calculating luminous energy W_{W2} of the second lamp bodies and luminous energy W_{W3} of the third lamp bodies;
making W_{W2}>W_{W02} and W_{W3}>W_{W03} when the brightness value I₀₂ is less than the brightness value Iᵣ₂;
making W_{W2}<W_{W02} and W_{W3}<W_{W03} when the brightness value I₀₂ is greater than the brightness value Iᵣ₂.
